(19) United States
(12) Patent Application Publication
(10) Pub. No.: US 2022/0240872 A1
(43) Pub. Date: Aug. 4, 2022

(54) REAL-TIME HEALTH DETECTION AND ALERT SYSTEM

(71) Applicants: Sudhir Shrikant Bhapkar, Jersey City, NJ (US); Priyanka Sudhir Bhapkar, Jersey City, NJ (US); Sarthak Sanjay Bhapkar, Pune (IN); Shivtej Vikram Gaikwad, Dhayari Pune (IN); Shivendra Sandeep Nimbalkar, Hadapsar Pune (IN); Yogesh Mohanrao Nigade, Pune (IN)

(72) Inventors: Sudhir Shrikant Bhapkar, Jersey City, NJ (US); Priyanka Sudhir Bhapkar, Jersey City, NJ (US); Sarthak Sanjay Bhapkar, Pune (IN); Shivtej Vikram Gaikwad, Dhayari Pune (IN); Shivendra Sandeep Nimbalkar, Hadapsar Pune (IN); Yogesh Mohanrao Nigade, Pune (IN)

(21) Appl. No.: 17/723,858

(22) Filed: Apr. 19, 2022

(51) Int. Cl. A61B 5/00; A61B 5/0205; A61B 5/024; G06V 20/56; H04W 4/029

(52) U.S. Cl. CPC A61B 5/747; A61B 5/746; A61B 5/7282; H04W 4/029; A61B 5/02438; G06V 20/56; A61B 5/02055

(57) ABSTRACT

The health detection and alert system comprises a user's computing device comprises a camera, an accelerometer sensor, a gyro sensor, a temperature sensor, a heart rate sensor and a GPS sensor; a user to detect real time health condition of a user, wherein the user interface is configured to: detect accident and dizzy falling upon comparing a threshold acceleration value and current acceleration value detected using the accelerometer sensor; detect vehicle accident upon processing captured image/video using a vehicle camera; detect real time critical health condition upon comparing threshold temperature and heart rate of the user with real time body temperature and heart rate of the user; and a processor to send an alert to a registered number along with rescue team if a user met with an accident or the health of the user is in critical condition using a communication module.

### TECHNICAL FIELD

The present disclosure relates to a real-time health detection and alert system, in more details, the system facilitates in detecting accidents and serious health issue and alerting the rescue team and relatives for the same.

### BACKGROUND

Computerized Disruption, Prescription, and Self-Prescription of Apps Advancement is a sign of improvements in clinical innovation, with a rich family that long goes before contemporary computerized interruption, for example, owing to cell phones and related advances. In 1995, Christensen and Bower authored the term troublesome innovation (later named problematic advancement) to portray the production of new business sectors in light of novel emanant advances in view of values that are unique in relation to that of existing business sectors.

Omnipresence, availability, and knowledge of cell phone innovation, joined with expanding general revenue in wellbeing and the increasing expense of clinician-driven medical services, may all add to the development of one such new disturbed market, to be specific, with regards to mHealth. Wellbeing customers may now autonomously search out mHealth applications to help with the finding or the board of ailments. Cell phone camera applications for wound consideration and amplifier applications for rest of the executives are instances of mHealth applications utilizing worked in sensors where analytic or therapy systems once confined to the domains of formal clinical discussion are presently open to laypersons for download and self-solution, comprising expected interruption, which bypasses customary clinician-started care and oversight

In the view of the forgoing discussion, it is clearly portrayed that there is a need to have a real-time health detection and alert system.

### BRIEF SUMMARY

The present disclosure seeks to provide a user-friendly installation system for real-time health detection and alert.

In an embodiment, a real-time health detection and alert system is disclosed. The system includes a user's computing device comprises a camera, an accelerometer sensor, a gyro sensor, a temperature sensor, a heart rate sensor and a GPS sensor. The system further includes a user interface installed on the user's computing device to detect real time health condition of a user, wherein the user interface is configured to: detect accident and dizzy falling upon comparing a threshold acceleration value and current acceleration value detected using the accelerometer sensor; detect vehicle accident upon processing captured image/video using a vehicle camera; detect real time critical health condition upon comparing threshold temperature and heart rate of the user with real time body temperature and heart rate of the user, wherein the real time body temperature is detected with the temperature sensor and heart rate is detected with the heart rate sensor. The system further includes a processor coupled to the user's computing device to send an alert to a registered number along with rescue team if a user met with an accident or the health of the user is in critical condition via one or more of the phone alert, SMS, and Email using a communication module of the user's computing device.

In another embodiment, the processor automatically send notifications to the nearest Trauma hospital center, nearby police stations and available fire brigade places along with their relatives if a user met with an accident or any disaster happened and their health is in critical condition.

In another embodiment, the alert comprises the real time health condition along with real time video/image and the user's current GEO location.

In another embodiment, the real time driving video is capture for 1-2 mins of continuous segmentation using the vehicle camera when the user interface is installed on the vehicle, which is further helpful for police and insurance companies to identify the cause of accident.

An object of the present disclosure is to provide a user interface that will have sensors to identify customers current health condition.

Another object of the present disclosure is to provide a user interface that can be installed in cars, in smart watches or in phones.

Another object of the present disclosure is to provide alerts if a user had an accident or any disaster happened and their health is in critical condition then it will automatically send notifications to the nearest Trauma hospital center also to their relatives by phone alert, SMS, Email.

Another object of the present disclosure is to provide an alert notification with the user's current GEO location and the health statistics.

Another object of the present disclosure is to provide an alert notification to nearby police stations or available fire brigade places.

Another object of the present disclosure is to provide a feature when installed in cars, to capture the driving video of 1-2 mins of continuous segmentation saved into cloud server which can be helpful for police and insurance companies to identify the cause.

Another object of the present disclosure is to send all these notifications automatically or manually.

Another object of the present disclosure is to save people's lives from disastrous life events like accidents or any natural disasters.

Yet another object of the present disclosure is to deliver an expeditious and cost-effective real-time health detection and alert system.

To further clarify advantages and features of the present disclosure, a more particular description of the disclosure will be rendered by reference to specific embodiments thereof, which is illustrated in the appended drawings. It is appreciated that these drawings depict only typical embodiments of the disclosure and are therefore not to be considered limiting of its scope. The disclosure will be described and explained with additional specificity and detail with the accompanying drawings.

### BRIEF DESCRIPTION OF FIGURES

These and other features, aspects, and advantages of the present disclosure will become better understood when the following detailed description is read with reference to the accompanying drawings in which like characters represent like parts throughout the drawings, wherein:

FIG. 1 illustrates a block diagram of a real-time health detection and alert system in accordance with an embodiment of the present disclosure.

Further, skilled artisans will appreciate that elements in the drawings are illustrated for simplicity and may not have necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION

For the purpose of promoting an understanding of the principles of the disclosure, reference will now be made to the embodiment illustrated in the drawings and specific language will be used to describe the same. It will nevertheless be understood that no limitation of the scope of the disclosure is thereby intended, such alterations and further modifications in the illustrated system, and such further applications of the principles of the disclosure as illustrated therein being contemplated as would normally occur to one skilled in the art to which the disclosure relates.

It will be understood by those skilled in the art that the foregoing general description and the following detailed description are exemplary and explanatory of the disclosure and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this disclosure belongs. The system, methods, and examples provided herein are illustrative only and not intended to be limiting.

Embodiments of the present disclosure will be described below in detail with reference to the accompanying drawings.

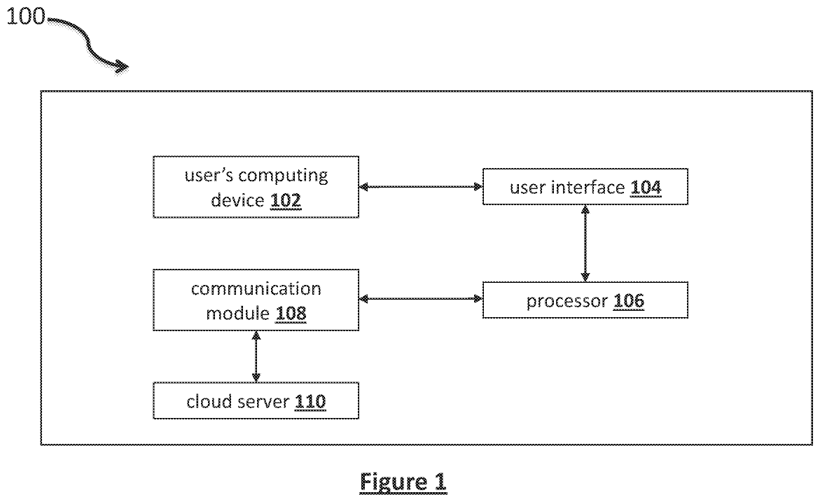

Referring to FIG. 1, a block diagram of a real-time health detection and alert system is illustrated in accordance with an embodiment of the present disclosure. The system 100 includes a user's computing device 102 comprises a camera, an accelerometer sensor, a gyro sensor, a temperature sensor, a heart rate sensor and a GPS sensor, and the like.

In an embodiment, a user interface 104 is installed on the user's computing device 102 to detect real time health condition of a user, wherein the user interface 104 is configured to detect accident and dizzy falling upon comparing a threshold acceleration value and current acceleration value detected using the accelerometer sensor. Then, detect vehicle accident upon processing captured image/video using a vehicle camera. Then, detect real time critical health condition upon comparing threshold temperature and heart rate of the user with real time body temperature and heart rate of the user, wherein the real time body temperature is detected with the temperature sensor and heart rate is detected with the heart rate sensor.

In an embodiment, a processor 106 is coupled to the user's computing device 102 to send an alert to a registered number along with rescue team if a user met with an accident or the health of the user is in critical condition via one or more of the phone alert, SMS, and Email using a communication module 108 of the user's computing device 102.

In another embodiment, the processor 106 automatically send notifications to the nearest Trauma hospital center, nearby police stations and available fire brigade places along with their relatives if a user met with an accident or any disaster happened and their health is in critical condition.

In another embodiment, the alert comprises the real time health condition along with real time video/image and the user's current GEO location.

In another embodiment, the real time driving video is capture for 1-2 mins of continuous segmentation using the vehicle camera when the user interface 104 is installed on the vehicle, which is further helpful for police and insurance companies to identify the cause of accident. The captured driving video of 1-2 mins of continuous segmentation is saved into cloud server 110 which can be helpful for police and insurance companies to identify the cause.

In excess of 2,000,000 wellbeing and prosperity applications (user interface 104) are accessible from the Apple and Google application stores. Some applications utilize cell phone sensors to produce wellbeing information. Clinicians and patients can observe data with respect to protected and powerful portable wellbeing (mHealth) applications in outsider organized mHealth application libraries. These autonomous Web-based archives guide application choice from confided in records, yet do they offer applications utilizing pervasive, minimal expense cell phone sensors to further develop wellbeing? This study expected to distinguish the kinds of underlying cell phone sensors utilized in applications recorded on organized wellbeing application libraries, the scope of medical issues these applications address, and the cross-stage accessibility of the applications.

This deliberate study explored three such storehouses (National Health Service Apps Library, AppScript, and MyHealthApps), surveying the accessibility of applications utilizing cell phone sensors for the analysis or treatment of medical issues.

Cell phone working frameworks upheld by included applications are noted, surveying the accessibility of these applications by clients of the Apple iOS and Google Android telephone types. The accessibility of included applications on publicized stages is affirmed by following connections promoted by every vault to investigate the Apple and Google application store application postings for applications. Applications are not downloaded or tried.

Application postings included as in scope by the lead analyst are then explored by the exploration group. Low numbers across result bunches blocked thorough measurable investigation. Unmistakable measurements are utilized where fitting to delineate outcomes and to permit examination between various libraries proportionate to separate library size.

Persistent mHealth application library curation, clinical gadget guideline imperatives, and cross-stage contrasts in cell phone sensor structures may all add to the noticed restricted accessibility of mHealth applications utilizing worked-in telephone sensors in organized mHealth application libraries.

Notwithstanding, more endeavors are expected to build the quantity of such applications on organized records, as they offer effectively open minimal expense choices to help individuals in overseeing clinical circumstances.

In one embodiment, an aggregate of 18 such applications are distinguished and remembered for this study, addressing 1.11% ( 7/677) to 3.2% ( 3/79) of all applications presented by the separate libraries inspected. Around 33% ( 7/18, 40%) of the distinguished applications offered cross-stage Apple and Android renditions, with a further 5=2% ( 10/20) simply committed to Apple and 13% ( 3/21) to Android.

In one embodiment, around one-fourth ( 4/18, 22.7%) of the recognized applications offered devoted demonstrative capacities, with a larger part including remedial ( 9/21, 50.5%) or consolidated usefulness ( 5/21, 28.7%). Cameras, contact screens, and amplifiers are the most often utilized implicit sensors. Wellbeing concerns tended to by these applications included respiratory, dermatological, neurological, and tension circumstances. camera ( 7/18, 39%) and contact screens ( 6/18, 33%) are the most often recognized cell phone sensors utilized (Table 4). Amplifiers and accelerometers (and cell phone speakers) are viewed as less every now and again involved sensors in the recognized applications. No GPS-based mHealth applications are recognized in this overview. MyHealthApps offered more camera-based applications than different libraries joined, while AppScript recorded more applications utilizing contact screens and amplifiers.

Respiratory ( 4/18, 22%), dermatological ( 4/18, 22%), neurological ( 3/18, 17%), nervousness ( 3/18, 17%), and visual wellbeing ( 2/18, 11%) are the dominating wellbeing concerns tended to by the recognized applications (Table 5). MyHealthApps and AppScript libraries recorded more applications tending to a more extensive scope of medical issue than the NHS Apps Library.

In one embodiment, the AppScript archive introduced more applications for respiratory-related conditions, concerning wheezing (n=2) and breathing activities (n=2). Both the applications utilizing worked-in sensors in the NHS Apps Library tended to the administration of tension.

The developed system facilitates a user interface 104 (app) that will have below human life saving features. This user interface 104 will have sensors to identify customer's current health conditions. If a user had an accident or any disaster happened and their health is in critical condition then it will automatically send notifications to the nearest Trauma hospital center also to their relatives by phone alert, SMS, Email. In this alert notification the system will send the user's current to GEO location and the health statistics. In such incidents, the system can also send automatic notification to nearby police stations or available fire brigade places. With this user interface 104 when installed in cars, includes a feature to capture the driving video of 1-2 mins of continuous segmentation which can be helpful for police and insurance companies to identify the cause. User interface 104 will have options to send these notifications automatically or manually. The user interface 104 can be installed in cars and in smart watches or in phones. This will help to save people's lives after an accident or any other disaster. An aggregate of 18 such applications are distinguished and remembered for this study, addressing 1.11% ( 7/677) to 3.2% ( 3/79) of all applications presented by the separate libraries inspected. Around 33% ( 7/18, 40%) of the distinguished applications offered cross-stage Apple and Android renditions, with a further 5=2% ( 10/20) simply committed to Apple and 13% ( 3/21) to Android. Around one-fourth ( 4/18, 22.7%) of the recognized applications offered devoted demonstrative capacities, with a larger part including remedial ( 9/21, 50.5%) or consolidated usefulness ( 5/21, 28.7%). Cameras, contact screens, and amplifiers are the most often utilized implicit sensors. Wellbeing concerns tended to by these applications included respiratory, dermatological, neurological, and tension circumstances.

The developed user interface 104 will have below human life saving features

1. This user interface 104 will have sensors to identify customers current health condition

2. If a user had an accident or any disaster happened and their health is in critical condition then it will automatically send notifications to the nearest Trauma hospital center also to their relatives by phone alert, SMS, Email.

3. In this alert notification, the system send the user's current to GEO location and the health statistics.

4. In such incidents, the system can also send automatic notification to nearby police stations or available fire brigade places.

5. With this user interface 104 when installed in cars, the system can also add a feature to capture the driving video of 1-2 mins of continuous segmentation which can be helpful for police and insurance companies to identify the cause.

6. User interface 104 will have options to send these notifications automatically or manually.

7. The user interface 104 can be installed in cars and in smart watches or in phones

8. This will help to save people's lives from disastrous life events like accidents or any natural disasters.

All applications tending to ailments utilizing worked in cell phone sensors to create wellbeing information are recognized utilizing the openly available hunt interfaces presented by every vault.

As no hunt rules are presented by these destinations for separating and distinguishing sensor-based applications, manual screening of the portrayals of all individual applications recorded by every site is directed by the lead scientist to evaluate for the utilization of inherent cell phone sensors.

Arranged library portrayals for distinguished applications are reviewed to classify the motivation behind the application as exclusively symptomatic, restorative, or a blend of both. Demonstrative applications are characterized as those that recognize the idea of a medical issue, rather than therapy applications, which offered highlights for ailment the board. Medical issue tended to be included applications are arranged in view of the portrayal given by every archive.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. A health detection and alert system, the system comprises:
a user computing device comprising a camera, an accelerometer sensor, a gyro sensor, a temperature sensor, a heart rate sensor, and a GPS sensor;
a user interface installed on the user computing device to detect real time health condition of a user, wherein the user interface is configured to:
detect accident and dizzy falling upon comparing a threshold acceleration value and current acceleration value detected using the accelerometer sensor;
detect vehicle accident upon processing captured image/video using a vehicle camera; and
detect real time critical health condition upon comparing threshold temperature and heart rate of the user with real time body temperature and heart rate of the user, wherein the real time body temperature is detected with the temperature sensor and heart rate is detected with the heart rate sensor; and
a processor coupled to the user's computing device to send an alert to a registered number along with rescue team if a user met with an accident or the health of the user is in critical condition via one or more of the phone alert, SMS, and Email using a communication module of the user's computing device.

2. The system of claim 1, wherein the processor automatically send notifications to the nearest Trauma hospital center, nearby police stations and available fire brigade places along with their relatives if a user met with an accident or any disaster happened and their health is in critical condition.

3. The system of claim 1, wherein the alert comprises the real time health condition along with real time video/image and the user's current GEO location.

4. The system of claim 1, wherein the real time driving video is capture for 1-2 mins of continuous segmentation using the vehicle camera when the user interface is installed on the vehicle, which is further helpful for police and insurance companies to identify the cause of accident.
